(19) United States
(12) Patent Application Publication
(10) Pub. No.: US 2026/0083746 A1
(43) Pub. Date: Mar. 26, 2026

(54) REDUCED DOSING REGIMENS FOR GLAUCOMA TREATMENT

(71) Applicant: Harrow IP, LLC, Nashville, TN (US)

(72) Inventor: Dennis Elias Saadeh, Nashville, TN (US)

(21) Appl. No.: 19/339,566

(22) Filed: Sep. 25, 2025

Related U.S. Application Data
(60) Provisional application No. 63/698,831, filed on Sep. 25, 2024.

(51) Int. Cl. A61K 31/5377; A61K 9/00; A61K 31/382; A61K 31/498; A61K 31/5575; A61K 47/02; A61K 47/10; A61K 47/12; A61K 47/26; A61P 27/06

(52) U.S. Cl. CPC A61K 31/5377; A61K 9/0048; A61K 31/382; A61K 31/498; A61K 31/5575; A61K 47/02; A61K 47/10; A61K 47/12; A61K 47/26; A61P 27/06

(57) ABSTRACT

Methods for treating open-angle glaucoma include transitioning a patient from a twice-per-day or more treatment regimen to a once-per-day treatment regimen. The once-per-day treatment include administering a single dose of a pharmaceutical composition including timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof, once per day.

### CROSS-REFERENCE TO RELATED APPLICATION

This application is related to and claims priority under 35 U.S.C. § 119 from U.S. Provisional Application No. 63/698,831 filed Sep. 25, 2024, titled “Composite Materials for Use in a Cathode Layers and Methods of Making the Same,” the entire contents of which are fully incorporated by reference herein for all purposes.

### FIELD OF THE DISCLOSURE

The present disclosure is directed to methods for treating glaucoma, including open-angle glaucoma, by administering a pharmaceutical composition comprising dorzolamide, brimonidine, timolol, and bimatoprost once per day.

### BACKGROUND

The burdens of glaucoma treatment including cost, ocular surface disease, periorbitopathy, and the inconvenience of dosing several times a day sometimes prompt physicians to discontinue part of a patient's regimen. In sub-Saharan Africa, the prevalence of glaucoma blindness is twice that of the rest of the world, and inability to afford medication is judged to be a major factor in adherence and outcomes.

What is needed are compositions and methods for treating glaucoma that require less frequent dosing.

### SUMMARY

Provided herein are methods for treating glaucoma comprising administering a composition only once per day. Generally, the methods comprise administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject in a single dose once per day. The subject may be previously administered a single dose of the pharmaceutical composition twice per day or more, and the method may further comprise reducing the frequency of the previous administration to once per day. In some aspects, reducing the frequency of administering the pharmaceutical composition to one time per day raises the diurnal intraocular pressure by no more than 0.4 mm Hg.

In some embodiments, the subject's mean intraocular pressure is statistically similar to the intraocular pressure of a patient treated with a pharmaceutical composition comprising latanoprost every other evening and a pharmaceutical composition comprising dorzolamide and timolol every morning. In some embodiments, the subject's mean intraocular pressure is statistically similar to the intraocular pressure of a patient treated with a pharmaceutical composition comprising latanoprost every evening and a pharmaceutical composition comprising dorzolamide and timolol two times per day.

In some embodiments, the concentration of timolol or a pharmaceutically acceptable salt thereof in the pharmaceutical composition is about 0.5% w/v; the concentration of brimonidine or a pharmaceutically acceptable salt thereof in the pharmaceutical composition is about 0.15% w/v; the concentration of dorzolamide or a pharmaceutically acceptable salt thereof in the pharmaceutical composition is about 2% w/v; and the concentration of bimatoprost or a pharmaceutically acceptable salt thereof in the pharmaceutical composition is about 0.01% w/v.

In some embodiments, the pharmaceutical composition is preservative-free. In some embodiments, the administration is topical administration via eye drops. In some embodiments, the concentration of timolol or a pharmaceutically acceptable salt thereof in the pharmaceutical composition is about 0.5% w/v. In some embodiments, the concentration of brimonidine or a pharmaceutically acceptable salt thereof in the pharmaceutical composition is about 0.15% w/v. In some embodiments, the concentration of dorzolamide or a pharmaceutically acceptable salt thereof in the pharmaceutical composition is about 2% w/v. In some embodiments, the concentration of bimatoprost or a pharmaceutically acceptable salt thereof in the pharmaceutical composition is about 0.01% w/v. In some embodiments, the pharmaceutical composition further comprises sodium citrate, sodium chloride, polysorbate 80, poloxamer 407, and water for injection.

Further provided herein are methods of treating glaucoma such as open angle glaucoma in a subject in need thereof. The methods generally comprise stopping administration of a pharmaceutical composition comprising latanoprost or a pharmaceutically acceptable salt thereof every evening and a pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. In some embodiments, the subject is being administered the pharmaceutical composition comprising latanoprost or a pharmaceutically acceptable salt thereof every evening and the pharmaceutical composition comprising dorzolamide or a pharmaceutically acceptable salt thereof and timolol or a pharmaceutically acceptable salt thereof two times per day prior for the treatment of open-angle glaucoma prior to stopping the administration.

In some embodiments, the concentration of timolol or a pharmaceutically acceptable salt thereof in the pharmaceutical composition is about 0.5% w/v; the concentration of brimonidine or a pharmaceutically acceptable salt thereof in the pharmaceutical composition is about 0.15% w/v; the concentration of dorzolamide or a pharmaceutically acceptable salt thereof in the pharmaceutical composition is about 2% w/v; and the concentration of bimatoprost or a pharmaceutically acceptable salt thereof in the pharmaceutical composition is about 0.01% w/v.

In some embodiments, the pharmaceutical composition is preservative-free. In some embodiments, the administration is topical administration via eye drops. In some embodiments, the concentration of timolol or a pharmaceutically acceptable salt thereof in the pharmaceutical composition is about 0.5% w/v. In some embodiments, the concentration of brimonidine or a pharmaceutically acceptable salt thereof in the pharmaceutical composition is about 0.15% w/v. In some embodiments, the concentration of dorzolamide or a pharmaceutically acceptable salt thereof in the pharmaceutical composition is about 2% w/v. In some embodiments, the concentration of bimatoprost or a pharmaceutically acceptable salt thereof in the pharmaceutical composition is about 0.01% w/v. In some embodiments, the pharmaceutical composition further comprises sodium citrate, sodium chloride, polysorbate 80, poloxamer 407, and water for injection.

Further provided herein is a method of reducing patient burden during treatment for glaucoma in a subject in need thereof. The methods generally comprise stopping administration of a pharmaceutical composition comprising latanoprost or a pharmaceutically acceptable salt thereof every other evening and a pharmaceutical composition comprising dorzolamide or a pharmaceutically acceptable salt thereof and timolol or a pharmaceutically acceptable salt thereof every morning; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. In some embodiments, the subject is being administered the pharmaceutical composition comprising latanoprost or a pharmaceutically acceptable salt thereof every evening and the pharmaceutical composition comprising dorzolamide or a pharmaceutically acceptable salt thereof and timolol or a pharmaceutically acceptable salt thereof two times per day prior for the treatment of open-angle glaucoma prior to stopping the administration.

In some embodiments, the concentration of timolol or a pharmaceutically acceptable salt thereof in the pharmaceutical composition is about 0.5% w/v; the concentration of brimonidine or a pharmaceutically acceptable salt thereof in the pharmaceutical composition is about 0.15% w/v; the concentration of dorzolamide or a pharmaceutically acceptable salt thereof in the pharmaceutical composition is about 2% w/v; and the concentration of bimatoprost or a pharmaceutically acceptable salt thereof in the pharmaceutical composition is about 0.01% w/v.

In some embodiments, the pharmaceutical composition is preservative-free. In some embodiments, the administration is topical administration via eye drops. In some embodiments, the concentration of timolol or a pharmaceutically acceptable salt thereof in the pharmaceutical composition is about 0.5% w/v. In some embodiments, the concentration of brimonidine or a pharmaceutically acceptable salt thereof in the pharmaceutical composition is about 0.15% w/v. In some embodiments, the concentration of dorzolamide or a pharmaceutically acceptable salt thereof in the pharmaceutical composition is about 2% w/v. In some embodiments, the concentration of bimatoprost or a pharmaceutically acceptable salt thereof in the pharmaceutical composition is about 0.01% w/v. In some embodiments, the pharmaceutical composition further comprises sodium citrate, sodium chloride, polysorbate 80, poloxamer 407, and water for injection.

### BRIEF DESCRIPTION OF THE FIGURES

The present disclosure may be understood by reference to the following detailed description taken in conjunction with the drawings briefly described below. It is noted that, for purposes of illustrative clarity, certain elements in the drawings may not be drawn to scale.

FIG. 1 shows a flow chart of a trial design for the trial described in Example 1.

FIG. 2 shows the mean IOP of each treatment, as measured at each phase and visit time, of the trial described in Example 1.

FIG. 3 shows the mean IOP of each treatment as measured at each phase and visit time of the trial described in Example 1.

FIG. 4 shows the Standard Patient Evaluation of Eye Dryness (SPEED) dry eye score by treatment and phase of the trial described in Example 1.

FIG. 5 shows an Euler diagram of the 68 patients who used systematized dosing and the 32 who did not in the trial described in Example 1. The ellipses show the number within each group who missed doses or found dosing difficult.

### DETAILED DESCRIPTION

Before various aspects of the present invention are disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity and in another example, a numerical range of “about 50 mg/mL to about 80 mg/mL” should also be understood to provide support for the range of “50 mg/mL to 80 mg/mL.”

As used herein, the term “subject” refers to human. The subject may be an adolescent or an adult. The subject may be male or female.

In this disclosure, the terms “including,” “containing,” and/or “having” are understood to mean comprising, and are open ended terms.

As used herein, the term “glaucoma” refers to a disease involving eye pressure increases that can lead to permanent vision loss and blindness. Unless otherwise specified, the term “glaucoma” refers to all types of glaucoma, including open-angle glaucoma, angle-closure glaucoma, secondary glaucoma, congenital glaucoma, neurovascular glaucoma, and any other types or subtypes of glaucoma known in the art.

Described herein are methods for treating open angle glaucoma in a subject in need thereof comprising administering a pharmaceutical composition to the subject once per day. The pharmaceutical composition administered to the subject comprises timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof. It has been surprisingly found that administration of a single dose of the pharmaceutical composition described herein is as effective as similar twice-per-day treatments using other pharmaceutical compositions. Transitioning patients from twice-per-day or more frequent dosing regimens to the once-per-day treatment described herein may improve patient compliance with medication and ultimately improve treatment outcomes.

The pharmaceutical composition may be administered to the subject once per day via topical administration. Preferably, the pharmaceutical composition is administered once per day via eye drops.

Administration of a single dose of the pharmaceutical composition once per day as described herein may result in achieving a mean intraocular pressure of a patient treated with other pharmaceutical compositions for the treatment of glaucoma, including open angle glaucoma. In some embodiments, administration of a single dose of the pharmaceutical composition once per day as described herein may result in the subject's mean intraocular pressure being statistically similar to the intraocular pressure of a subject treated with 1) a topical ophthalmic pharmaceutical composition comprising latanoprost every other evening and 2) a topical ophthalmic pharmaceutical composition comprising dorzolamide and timolol every morning. In some embodiments, administration of a single dose of the pharmaceutical once per day as described herein may result in the subject's mean intraocular pressure being statistically similar to the intraocular pressure of a subject treated with 1) a topical ophthalmic pharmaceutical composition comprising latanoprost every evening and 2) a topical ophthalmic pharmaceutical composition comprising dorzolamide and timolol two times per day.

In some embodiments, reducing the frequency of administering a single dose of the pharmaceutical composition to once per day as described herein may raise the diurnal intraocular pressure of the subject by no more than 0.4 mm Hg. For example, reducing the frequency of administering a single dose of the pharmaceutical composition to once per day as described herein may raise the diurnal intraocular pressure of the subject by no more than 0.4 mm Hg, no more than 0.3 mm Hg, no more than 0.2 mm Hg, or no more than 0.1 mm Hg. In some embodiments, reducing the frequency of administering a single dose of the pharmaceutical composition to once per day as described herein may not raise the diurnal intraocular pressure of the subject.

In some embodiments, the subject is previously administered a single dose of the pharmaceutical composition twice per day or more. In such embodiments, the method further comprises reducing the frequency of the previous administration frequency from twice per day or more to once per day. The reduction in frequency may be immediate or gradual. For example, if the subject was previously administered a single dose of the pharmaceutical composition three times per day, the subject may transition immediately to administration of the pharmaceutical composition once per day. Alternatively, the subject may first reduce the dosing frequency to administration of the pharmaceutical composition twice per day for a period of time before finally reducing the dosing frequency to administration of the pharmaceutical composition once per day.

The pharmaceutical composition used in the methods of the present disclosure includes timolol, brimonidine, dorzolamide, and bimatoprost. As discussed further herein, the pharmaceutical composition is suitable for administration once per day for the treatment of glaucoma, including open angle glaucoma. The pharmaceutical composition is suitable for topical administration (e.g., eye drops).

The pharmaceutical composition includes timolol or a pharmaceutically acceptable salt thereof. In preferred embodiments, the timolol comprises timolol maleate. Timolol is a propanolamine derivative used to treat glaucoma via the reduction of intraocular pressure.

The timolol or a pharmaceutically acceptable salt thereof may be present in the pharmaceutical composition in a concentration from about 0.1% w/v to about 1% w/v. For example, the timolol or a pharmaceutically acceptable salt thereof may be present in the pharmaceutical composition in a concentration from about 0.1% w/v to about 0.5% w/v, about 0.5% w/v to about 1% w/v, about 0.1% w/v to about 0.9% w/v, about 0.2% w/v to about 0.8% w/v, about 0.3% w/v to about 0.7% w/v, or about 0.4% w/v to about 0.6% w/v. As another example, the timolol or a pharmaceutically acceptable salt thereof may be present in the pharmaceutical composition in a concentration of about 0.1% w/v, 0.15% w/v, 0.2% w/v, 0.25% w/v, 0.3% w/v, 0.35% w/v, 0.4% w/v, 0.45% w/v, 0.5% w/v, 0.55% w/v, 0.6% w/v, 0.65% w/v, 0.7% w/v, 0.75% w/v, 0.8% w/v, 0.85% w/v, 0.9% w/v, 0.95% w/v, or about 1% w/v. In an exemplary embodiment, the timolol or a pharmaceutically acceptable salt thereof may be present in the pharmaceutical composition in a concentration of about 0.5% w/v.

The pharmaceutical composition includes brimonidine or a pharmaceutically acceptable salt thereof. In preferred embodiments, the brimonidine includes brimonidine tartrate. Brimonidine is an imidazole derivative used to treat glaucoma via the reduction of intraocular pressure. Brimonidine works by acting on blood vessels causing constriction, which leads to a decrease in the production of aqueous humor, and also increases the outflow of aqueous humor.

The brimonidine or a pharmaceutically acceptable salt thereof may be present in the pharmaceutical composition in a concentration from about 0.01% w/v to about 0.3% w/v. For example, the brimonidine or a pharmaceutically acceptable salt thereof may be present in the pharmaceutical composition in a concentration from about 0.01% w/v to about 0.1% w/v, about 0.1% w/v to about 0.2% w/v, or about 0.2% w/v to about 0.3% w/v, about 0.11% w/v to about 0.19% w/v, about 0.12% w/v to about 0.18% w/v, about 0.13% w/v to about 0.17% w/v, or about 0.14% w/v to about 0.16% w/v. As another example, the brimonidine or a pharmaceutically acceptable salt thereof may be present in the pharmaceutical composition in a concentration of about 0.01% w/v, 0.05% w/v, 0.1% w/v, 0.11% w/v, 0.12% w/v, 0.13% w/v, 0.14% w/v, 0.15% w/v, 0.16% w/v, 0.17% w/v, 0.18% w/v, 0.19% w/v, 0.2% w/v, 0.25% w/v, or about 0.3% w/v. In an exemplary embodiment, the brimonidine or a pharmaceutically acceptable salt thereof may be present in the pharmaceutical composition in a concentration of about 0.15% w/v.

The pharmaceutical composition includes bimatoprost or a pharmaceutically acceptable salt thereof. Bimatoprost is used to treat glaucoma via reduction of intraocular pressure. Bimatoprost mimics the effects of endogenous prostamides reducing intraocular pressure by increasing outflow of aqueous humor through the trabecular meshwork and uveoscleral routes.

The bimatoprost or a pharmaceutically acceptable salt thereof may be present in the pharmaceutical composition in a concentration from about 0.001% w/v to about 0.1% w/v. For example, the bimatoprost may be present in the pharmaceutical composition in a concentration from about 0.001% w/v to about 0.01% w/v, about 0.01% w/v to about 0.1% w/v, about 0.005% w/v to about 0.05% w/v, about 0.005% w/v to about 0.2% w/v, about 0.005% w/v to about 0.15% w/v, about 0.006% w/v to about 0.014% w/v, about 0.007% w/v to about 0.013% w/v, about 0.008% w/v to about 0.012% w/v, or about 0.009% w/v to about 0.011% w/v. As another example, the bimatoprost may be present in the pharmaceutical composition in a concentration of about 0.001% w/v, 0.002% w/v, 0.003% w/v, 0.004% w/V, 0.005% w/v, 0.006% w/v, 0.007% w/v, 0.008% w/v, 0.009% w/v, 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v. In an exemplary embodiment, the bimatoprost may be present in the pharmaceutical composition in a concentration of about 0.01% w/v.

The pharmaceutical composition includes dorzolamide or a pharmaceutically acceptable salt thereof. In preferred embodiments, the dorzolamide includes dorzolamide hydrochloride. Dorzolamide is used to treat glaucoma via the inhibition of the enzyme carbonic anhydrase, which reduces the amount of aqueous humor produced in the eye thereby decreasing intraocular pressure.

The dorzolamide or a pharmaceutically acceptable salt thereof may be present in the pharmaceutical composition in a concentration from about 0.1% w/v to about 5% w/v. For example, the dorzolamide or a pharmaceutically acceptable salt thereof may be present in the pharmaceutical composition in a concentration from about 0.1% w/v to about 1% w/v, about 1% w/v to about 5% w/v, about 0.5% w/v to about 5% w/v, about 1% w/v to about 5% w/v, about 1% w/v to about 4% w/v, or about 1% w/v to about 3% w/v. As another example, the dorzolamide or a pharmaceutically acceptable salt thereof may be present in the pharmaceutical composition in a concentration of about 0.1% w/v, 0.5% w/v, 1% w/v, 1.1% w/v, 1.2% w/v, 1.3% w/v, 1.4% w/v, 1.5% w/v, 1.6% w/v, 1.7% w/v, 1.8% w/v, 1.9% w/v, 2% w/v, 2.1% w/v, 2.2% w/v, 2.3% w/v, 2.4% w/v, 2.5% w/v, 2.6% w/v, 2.7% w/v, 2.8% w/v, 2.9% w/v, 3% w/v, 3.5% w/v, 4% w/v, 4.5% w/v, or about 5% w/v. In an exemplary embodiment, the dorzolamide may be present in the pharmaceutical composition in a concentration of about 2% w/v.

Preferably, the pharmaceutical composition used in the methods described herein is preservative free; stated another way, in some embodiments the pharmaceutical composition used in the methods described herein contains less than 0.001% preservative or does not include any preservative. As used herein, the term “preservative” includes any substance that functions solely to prevent or slow the degradation of the pharmaceutical composition or of one or more active ingredients in the pharmaceutical composition. Examples of preservatives within the scope of the present disclosure include benzalkonium chloride, . . . .

The pharmaceutical composition used in the methods described herein may further include one or more excipients. The excipient(s) may include a buffer, an isotonicity agent, a wetting agent, a pH modifier, or any combination thereof.

The excipient may include a buffer. The buffer functions to stabilize the pH of the pharmaceutical composition. The buffer may include a citrate buffer (e.g., sodium citrate), a phosphate buffer, a borate buffer, a tris buffer, or any other buffer known in the art. In an exemplary embodiment, the buffer includes sodium citrate.

The buffer may be present in the pharmaceutical composition in an amount from about 1 mg/ml to about 2 mg/mL. For example, the buffer may be present in the pharmaceutical composition in an amount from about 1 mg/mL to about 1.2 mg/mL, about 1 mg/mL to about 1 mg/mL to about 1.4 mg/mL, about 1 mg/mL to about 1.6 mg/mL, about 1 mg/mL to about 1.8 mg/mL, about 1 mg/mL to about 2 mg/mL, about 1.2 mg/mL to about 1.4 mg/mL, about 1.2 mg/mL to about 1.6 mg/mL, about 1.2 mg/mL to about 1.8 mg/mL, about 1.2 mg/mL to about 2 mg/mL, about 1.4 mg/mL to about 1.6 mg/mL, about 1.4 mg/mL to about 1.8 mg/mL, about 1.4 mg/mL to about 2 mg/mL, about 1.6 mg/mL to about 1.8 mg/mL, about 1.6 mg/mL to about 2 mg/mL, or about 1.8 mg/mL to about 2 mg/mL. As another example, the buffer may be present in the pharmaceutical composition in an amount of about 1 mg/mL, 1.1 mg/ml, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or about 2 mg/mL. In an exemplary embodiment, the buffer is present in the pharmaceutical composition in an amount of about 1.5 mg/mL.

The pharmaceutical composition may include an isotonicity agent. The isotonicity agent may include sodium chloride, boric acid, propylene glycol, mannitol or other isotonicity agents known in the art. In an exemplary embodiment, the isotonicity agent includes sodium chloride.

The isotonicity agent may be present in the pharmaceutical composition in an amount from about 1 mg/mL to about 10 mg/mL. For example, the isotonicity agent may be present in the pharmaceutical composition in an amount from about 1 mg/ml to about 2 mg/mL, about 1 mg/mL to about 4 mg/mL, about 1 mg/mL to about 6 mg/mL, about 1 mg/mL to about 8 mg/mL, about 1 mg/mL to about 10 mg/mL, about 2 mg/mL to about 4 mg/mL, about 2 mg/mL to about 6 mg/mL, about 2 mg/mL to about 8 mg/mL, about 2 mg/mL to about 10 mg/mL, about 4 mg/mL to about 6 mg/mL, about 4 mg/mL to about 8 mg/mL, about 4 mg/mL to about 10 mg/mL, about 6 mg/mL to about 8 mg/mL, about 6 mg/mL to about 10 mg/mL, or about 8 mg/mL to about 10 mg/mL. As another example, the isotonicity agent may be present in the pharmaceutical composition in an amount of about 1 mg/ml, 1.5 mg/mL, 2 mg/mL, 2.5 mg/ml, 3 mg/ml, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, or about 10 mg/mL. In an exemplary embodiment, the isotonicity agent may be present in the pharmaceutical composition in an amount of about 3.54 mg/mL.

The pharmaceutical composition may include a wetting agent. The wetting agent may include a polysorbate (e.g., polysorbate 80), a poloxamer (e.g., poloxamer 407), hydroxypropyl methylcellulose, carboxymethylcellulose, polyethylene glycol, propylene glycol, glycerin, or other wetting agents known in the art. In an exemplary embodiment, the wetting agent includes polysorbate 80, poloxamer 407, or a combination thereof.

The wetting agent may be present in the pharmaceutical composition in an amount from about 0.0001 mg/mL to about 5 mg/mL. For example, the wetting agent may be present in the pharmaceutical composition in an amount from about 0.0001 mg/ml to about 0.001 mg/mL, about 0.0001 mg/mL to about 0.01 mg/mL, about 0.0001 mg/mL to about 0.1 mg/mL, about 0.0001 mg/mL to about 1 mg/mL, about 0.0001 mg/mL to about 5 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 5 mg/mL, about 0.01 mg/mL to about 0.1 mg/mL, about 0.01 mg/mL to about 1 mg/mL, about 0.01 mg/mL to about 5 mg/mL, about 0.1 mg/mL to about 1 mg/mL, about 0.1 mg/mL to about 5 mg/mL, or about 1 mg/mL to about 5 mg/mL. As another example, the wetting agent may be present in the pharmaceutical composition in an amount from about 0.0001 mg/mL, 0.00025 mg/mL, 0.0005 mg/mL, 0.00075 mg/mL, 0.001 mg/mL, 0.0025 mg/mL, 0.005 mg/mL, 0.0075 mg/mL, 0.01 mg/mL, 0.025 mg/mL, 0.05 mg/mL, 0.075 mg/mL, 0.1 mg/mL, 0.25 mg/mL, 0.5 mg/mL, 0.75 mg/mL, 1 mg/mL, 2 mg/ml, 3 mg/mL, 4 mg/mL, or about 5 mg/mL. In an exemplary embodiment, the wetting agent comprises polysorbate 80 in an amount of about 0.0005 mg/mL and poloxamer 407 in an amount of about 1 mg/mL.

The pharmaceutical composition may include a pH modifier. The pH modifier may include sodium hydroxide or hydrochloric acid. The pH modifier may be added dropwise to the pharmaceutical composition to adjust the pH to a desired value.

The pharmaceutical composition may have a pH from about 4.5 to about 7.5. For example, the pharmaceutical composition may have a pH from about 4.5 to about 5.5, about 4.5 to about 6.5, about 4.5 to about 7.5, about 5.5 to about 6.5, about 5.5 to about 7.5, or about 6.5 to about 7.5. As another example, the pharmaceutical composition may have a pH of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or about 7.5.

The pharmaceutical composition may further include a carrier. The carrier may include water, such as deionized water or sterile water for injection.

In an exemplary embodiment, the pharmaceutical composition is the pharmaceutical composition described in Table 1:

TABLE 1

| Ingredient | Concentration Range | Preferred Concentration | |
|---|---|---|---|
| Timolol or a | about 0.1% w/v to about 1% | 0.5% | w/v |
| pharmaceutically acceptable | w/v | | |
| salt thereof | | | |
| Brimonidine or a | about 0.01% w/v to about | 0.15% | w/v |
| pharmaceutically acceptable | 0.3% w/v | | |
| salt thereof | | | |
| Bimatoprost or a | about 0.001% w/v to about | 0.01% | w/v |
| pharmaceutically acceptable | 0.1% w/v | | |
| salt thereof | | | |
| Dorzolamide or a | about 0.1% w/v to about 5% | 2% | w/v |
| pharmaceutically acceptable | w/v | | |
| salt thereof | | | |
| Sodium citrate | about 1 mg/mL to about 2 | 1.5 | mg/mL |
| | mg/mL | | |
| Sodium chloride | about 1 mg/mL to about 10 | 3.54 | mg/mL |
| | mg/mL | | |
| Polysorbate 80 | about 0.0001 mg/mL to | 0.0005 | mg/mL |
| | about 5 mg/mL | | |
| Poloxamer 407 | about 0.0001 mg/mL to | 1 | mg/mL |

| | | |
|---|---|---|
| | about 5 mg/mL | |
| Hydrochloric acid/ | As needed | As needed |
| Sodium hydroxide | | |
| Water for injection | q.s. 1 g/mL | q.s. 1 g/mL |

Further provided herein are methods for treating glaucoma comprising stopping administration of a pharmaceutical composition comprising latanoprost or a pharmaceutically acceptable salt thereof every evening and a pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. Thus, prior to the administration of the once-per-day composition, the subject is being administered the pharmaceutical composition comprising latanoprost or a pharmaceutically acceptable salt thereof every evening and the pharmaceutical composition comprising dorzolamide or a pharmaceutically acceptable salt thereof and timolol or a pharmaceutically acceptable salt thereof two times per day prior for the treatment of open-angle glaucoma prior to stopping the administration. Stated another way, the methods comprise transitioning a subject being administered a twice-per-day treatment regimen to a once-per-day treatment regimen as described herein. The pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof administered once per day may be any of the pharmaceutical compositions described hereinabove.

Further provided herein are methods for treating glaucoma comprising stopping administration of a pharmaceutical composition comprising latanoprost or a pharmaceutically acceptable salt thereof every other evening and a pharmaceutical composition comprising dorzolamide or a pharmaceutically acceptable salt thereof and timolol or a pharmaceutically acceptable salt thereof every morning; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. The pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof administered once per day may be any of the pharmaceutical compositions described hereinabove.

Further provided herein are methods of reducing patient burden of a subject undergoing treatment for glaucoma. As used herein, “patient burden” refers to the burden on the patient to self-administer medication. Thus, a subject undergoing treatment requiring administration of multiple medication dosage forms or a subject undergoing treatment requiring administration of a medication multiple times per day would each have a higher patient burden as compared to a subject undergoing treatment requiring administration of a single dosage form once per day.

In some embodiments, the methods generally comprise transitioning a patient from a twice-per-day or more dosing regimen to a once-per day dosing regimen, wherein the once-per-day dosing regimen includes administering a pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof once per day to the subject.

In some embodiments, the methods generally comprise stopping administration of a pharmaceutical composition comprising latanoprost or a pharmaceutically acceptable salt thereof every evening and a pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. The pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof administered once per day may be any pharmaceutical composition described herein.

In some embodiments, the methods generally comprise stopping administration of a pharmaceutical composition comprising latanoprost or a pharmaceutically acceptable salt thereof every other evening and a pharmaceutical composition comprising dorzolamide or a pharmaceutically acceptable salt thereof and timolol or a pharmaceutically acceptable salt thereof every morning; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day. The pharmaceutical composition comprising dorzolamide or a pharmaceutically acceptable salt thereof and timolol or a pharmaceutically acceptable salt thereof two times per day; and administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof administered once per day may be any pharmaceutical composition described herein.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations, or methods of the disclosure may be made without departing from the spirit of the disclosure and the scope of the appended claims. Definitions of the variables in the structures in the schemes herein are commensurate with those of corresponding positions in the formulae presented herein.

### Example 1

A randomized crossover trial studying 63 eyes of 32 open angle glaucoma patients previously treated with selective laser trabeculoplasty (SLT) was performed to determine if less frequent dosing reduce treatment burdens without sacrificing efficacy. Patients were surveyed about adherence to every other day (QOD) dosing and trialed IOP control using latanoprost/dorzolamide-timolol at standard and half-frequency dosing, comparing also to a once daily preservative-free quadruple medication. In phase 1, patients used latanoprost every other evening/dorzolamide-timolol every morning (lat QOD/dor-tim QAM). In phase 2, patients were randomly assigned to either latanoprost every evening/dorzolamide-timolol twice daily (lat QPM/dor-tim BID) or every morning use of timolol-brimonidine-dorzolamide-bimatoprost (tim-brim-dor-bim QAM). In phase 3, patients were crossed over to the regimen not used in phase 2. After using a treatment for 3 weeks, Goldmann and pneumotonometry measurements were made at 8:00-9:00 and 16:00-17:00. A separate group of 101 patients not participating in the trial was surveyed about the difficulty of adhering to a QOD regimen.

Results: There was no significant IOP difference between the three dosing regimens. The mean IOPs (mm Hg with 95% confidence intervals) were: lat QOD/dor-tim QAM 12.3 mmHg (11.5-13.1), lat QPM/dor-tim BID 12.4 mmHg (11.6/13.2), and tim-brim-dor-bim QAM 12.1 mmHg (11.3-12.9). Comparing the reduced frequency regimens to a standard regimen of lat QPM/dor-tim BID, it is 95% probable that half frequency dosing would raise IOP<0.6 mm Hg and that the quadruple medication would raise IOP<0.4 mm Hg. Of patients surveyed about adherence to QOD dosing, 68% reported that they had systematized dosing, usually with a calendar; 81% said QOD dosing was “not difficult.” Patients without a dosing system were much likelier to report difficulty with the regimen (p<0.001) or missing a dose in the previous month (p=0.02).

Conclusion: For patients previously treated with SLT, regimens of either lat QOD/dor-tim QAM (1-2 drops/day) or preservative-free tim-brim-dor-bim QAM (1 drop/day) were non-inferior to lat QPM/dor-tim BID (3 drops/day). Most patients systematize QOD dosing, and those doing so report good adherence. Reduced dosing regimens could reduce treatment burden without compromising efficacy.

For patients unable to tolerate or afford a regimen, it was hypothesized that IOP could be more effectively controlled by reducing the dosing frequency of several medications instead of completely discontinuing a single medication. A prospective randomized cross-over trial to measure the IOP impact of dosing latanoprost/dorzolamide-timolol at half-frequency, with additional comparison to a single morning drop of timolol-brimonidine-dorzolamide-bimatoprost (tim-brim-dor-bim QAM) was performed. As an indicator of the difficulty of adhering to a QOD regimen, a separate group of patients who were using a QOD regimen were surveyed.

### Methods

The trial was performed as a prospective, single center, single blinded, randomized, cross-over, non-inferiority study. Participants were assigned by simple randomization into two groups, the allocation of which was unknown to the certified ophthalmic technician that collected the data.

In phase 1 of the trial, baseline morning and afternoon IOP measurements were taken while all subjects were taking their already established regimen of lat QOD/dor-tim QAM. For phase 2, participants were randomized to either use lat QPM/dor-tim BID or tim-brim-dor-bim QAM. After three weeks, morning and afternoon IOP measurements were taken for each participant. For phase 3, participants used whichever treatment regimen they had not yet received with diurnal IOP measurements being taken after three weeks. The IOP measurements were scheduled per participant convenience, without regard to whether a QOD drug had been used 1 or 2 days prior.

### Subjects

Trial inclusion criteria included adult, open angle glaucoma patients already using for at least 2 months the lat QOD/dor-tim QAM dosing regimen and previously treated with selective laser trabeculoplasty (SLT) within the previous 4 years. Exclusion criteria included use of ocular laser, surgery, or corticosteroids within the previous 3 months; logistical inability to attend the required 6 trial visits; inability to relax the eye muscles for accurate tonometry; inability to instill medications reliably; allergy to brimonidine; and a patient's preference not to participate.

A total of 6720 glaucoma patients were screened with the inclusion and exclusion criteria. After screening, 36 participants were enrolled in the trial. Of the 36 enrolled participants, one elected to include only one eye in the study, two withdrew due to adverse reactions to tim-brim-dor-bim QAM, one withdrew due to change of mind, and one discontinued due to logistical reasons. Ultimately, 32 participants completed the trial and data from 63 eyes were analyzed. The demographic characteristics of the trial participants are tabulated in Table 2.

TABLE 2
Trial Participant Characteristics

| | | Total | | Group 1 | | Group 2 | |
|---|---|---|---|---|---|---|---|
| Number | | 32 | | 16 | | 16 | |
| Sex | Male | 19 | (59.4%) | 9 | (56.3%) | 10 | (62.5%) |
| | Female | 13 | (40.6%) | 7 | (43.8%) | 6 | (37.5%) |
| Age | Range | 48-84 | | 58-84 | | 48-79 | |
| | Mean | 70.4 | | 73.4 | | 67.4 | |
| | Standard | 8.96 | | 7.93 | | 9.16 | |
| | deviation | | | | | | |
| Ancestry | African | 5 | (15.6%) | 3 | (18.7%) | 2 | (12.5%) |
| | Asian | 1 | (3.1%) | 1 | (6.2%) | 0 | |
| | European | 22 | (68.7%) | 12 | (75%) | 10 | (62.5%) |

| | | | | | |
|---|---|---|---|---|---|
| Latin American | 4 | (12.5%) | 0 | 4 | (25%) |

The QOD regimen survey participants were a separate group of consecutive patients using a prostaglandin QOD. (A random sampling of 104 prostaglandin-treated patients found that 100 were prescribed prostaglandin every other day.) Of 101 patients surveyed, one declined to participate. The average age of survey participants was 64.2 years and the predominant ancestry was European 56%, African 36%, and Latin American 8%.

### Assessments

IOP measurements for each participant were taken in the morning (8:00-9:00) and afternoon (16:00-17:00) during the Phase 1 baseline treatment (lat QOD/dor-tim QAM) and after 3 weeks of starting randomized interventions. All measurements were taken by the same certified ophthalmic assistant that was blinded to the assignment randomization. Prior to taking measurements, it was verified that the study medication had been used the morning of the visit (4 visits were rescheduled because the AM medication had been skipped). All measurements were made in duplicate with a pneumotonometer (Model 30, Reichert, Buffalo, NY) and Goldmann tonometer, using the same 2 devices throughout the study.

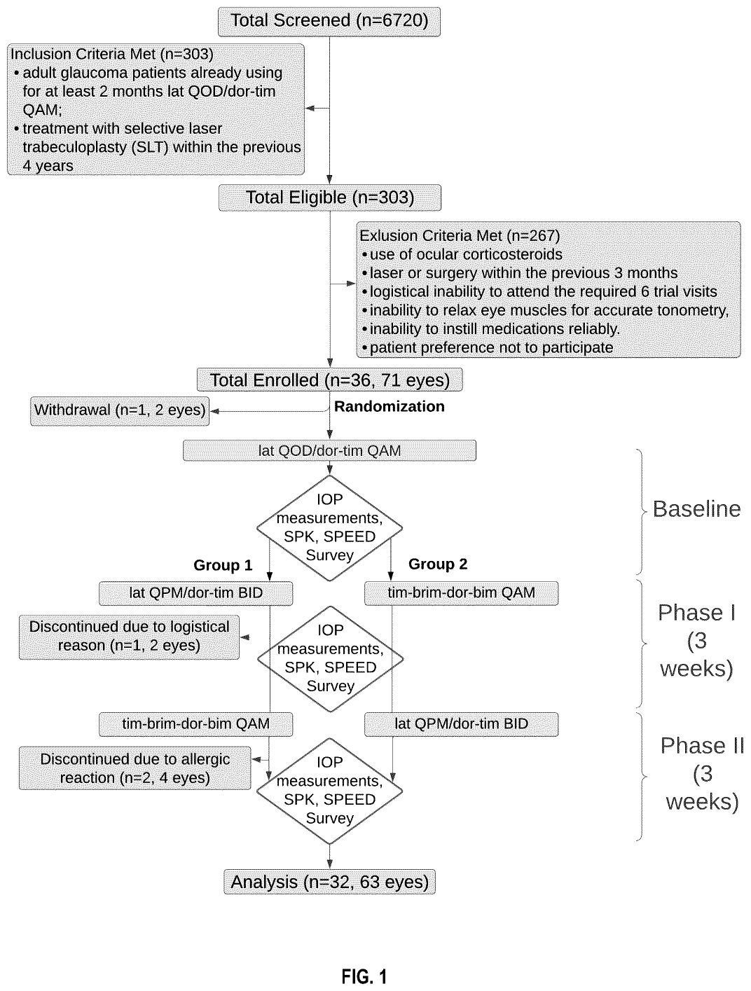

During morning measurements, superficial punctate keratitis (SPK) was graded on a scale of 0-4+ using slit lamp biomicroscopy to assess fluorescein staining and a Standard Patient Evaluation of Eye Dryness (SPEED) questionnaire was administered. The study design including enrollment, treatments and measurements is summarized in FIG. 1.

For survey participants, a physician inquired about difficulty of dosing, systematization of dosing and missed doses using the following script: “Many patients find an every other day medicine schedule difficult, because it's hard to remember whether or not the drop was taken yesterday. Or, they miss the drop several days in a row because it is not a daily habit. How is that for you—do you find that every other day is a bit difficult or quite difficult?”

Responses were categorized as: not at all difficult, a bit difficult, or quite difficult. If the patient stated, without prompting, that a system was used to track dosing, this fact was noted; otherwise, the patient was asked what system, if any, was used to track dosing. The patient was then asked whether 2 consecutive days of medicine had been missed in the prior month.

### Study Size, Baseline Peak/Trough, Statistical Analysis

Sample size planning was made from previously generated pilot data. In the pilot, patients were asked to use the second dose of dorzolamide-timolol a few hours before the afternoon IOP measurement, instead of in the evening. That trial showed no morning IOP difference, but there was an afternoon IOP difference with an overall mean 0.5 mm Hg (standard deviation 1.2) difference. The correlation between the standard dose and half dose across patients was estimated to be about r=0.7. It was calculated that 90% power to detect a non-inferiority margin of 1.0 mm Hg IOP difference would require 62 subjects. However, an interim analysis of the data from 63 eyes of 32 subjects revealed that the intended non-inferiority margin had already been demonstrated, so the study was halted. The likely explanation for reaching the non-inferiority endpoint sooner than expected is that use of an early afternoon dose of dorzolamide-timolol in the pilot (instead of conventional evening dosing) gave an optimistic picture of a full dose afternoon IOP.

In the pilot, all morning measurements of half-frequency dosing were made at trough (tafluprost used 2 nights earlier), and full dosing showed no advantage for morning IOP. Nevertheless, it was reasoned that if there were a difference between morning IOPs depending on whether the prostaglandin was used 1 or 2 nights previously, measuring only at trough would give a false picture of the average IOP with half-frequency dosing. Accordingly, all baseline measurements were scheduled per patient convenience and without regard to whether the last dose of latanoprost would be taken 1 or 2 nights before measurement, with the expectation that baseline measurements would then represent a reasonably random sampling of peak and trough states.

For the trial, Goldmann and pneumotonometry for each subject visit were treated as repeated measures. The Goldmann tonometer was calibrated with a weighted arm and the pneumotonometer was calibrated using the manufacturer's reference (a water column above a silicone applanation membrane), to ensure IOP data confidence. To account for the correlated data between left and right eyes of each enrolled subject, linear mixed effects models were run with eye nested beneath subject for random effects (software SPSS v28, IBM, USA).

Survey data was analyzed using the chi-square statistic.

### Results

### Trial: Primary Outcomes

It was found that despite the compounded quadruple agent (tim-brim-dor-bim QAM) giving an average IOP 0.25 mm Hg lower than the other 2 treatments (lat QOD/dor-tim QAM and lat QPM/dor-tim BID), there was no significant IOP difference between treatments at morning, afternoon, or considering both time points together (Table 3).

TABLE 3
Mean IOPs of each treatment

| | | Standard | 95% confidence interval | |
|---|---|---|---|---|
| Treatment | Mean IOP | error | Lower bound | Upper bound |
| lat QOD/dor-tim QAM | 12.34 | 0.395 | 11.5 | 13.1 |
| lat QPM/dor-tim BID | 12.38 | 0.396 | 11.6 | 13.2 |
| tim-brim-dor-bim QAM | 12.11 | 0.396 | 11.3 | 12.9 |

A pairwise comparison of the treatments showed that it is 95% likely that, compared to standard dosing of latanoprost/dorzolamide-timolol, halved dosing would give IOP<0.6 mm Hg higher, and one daily drop of the quadruple medication would give IOP<0.4 mm Hg higher (Table 4).

TABLE 4
Non-inferiority of reducing dosing treatments

| | | | | 95% confidence | |
|---|---|---|---|---|---|
| | | | | interval for difference | |
| Treatment | Mean IOP | Standard | | Lower | Upper |
| Comparison | difference | error | p-value | bound | bound |
| (half dose) − (full dose) | 0.04 | 0.31 | 0.89 | −0.66 | 0.58 |
| (quad) − (full dose) | −0.26 | 0.31 | 0.39 | −0.88 | 0.36 |

half dose = lat QOD/dor-tim QAM;
full dose = lat QPM/dor-tim BID;
quad = tim-brim-dor-bim QAM

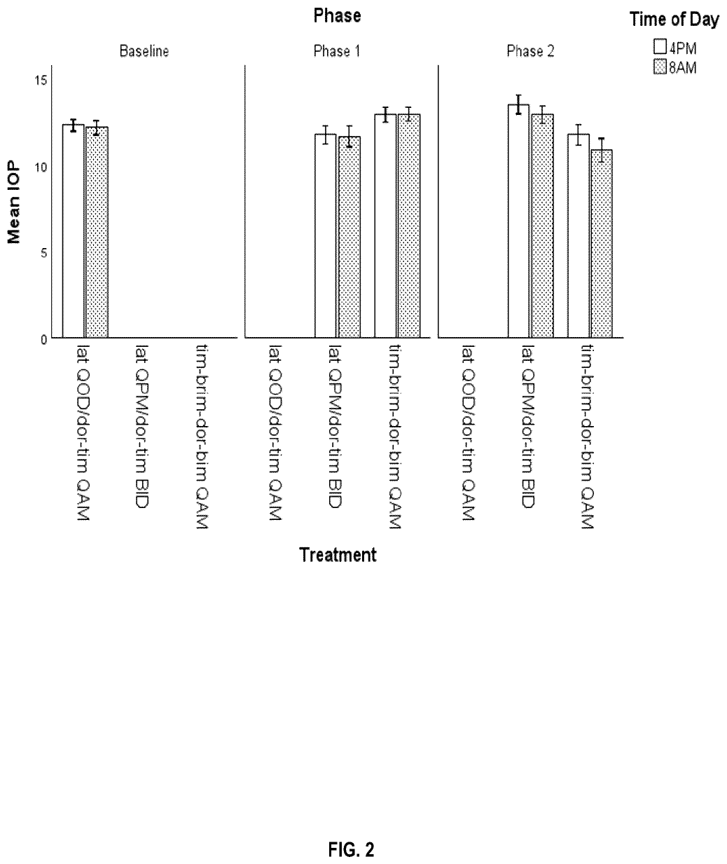

FIG. 2 shows the mean IOP of each treatment, as measured at each phase and visit time. The mean IOP of all morning visits was 12.1 mm Hg and that of afternoon visits was 12.4 mm Hg; the difference between morning and afternoon IOPs was not significant (p=0.06).

### Trial: Secondary Outcomes

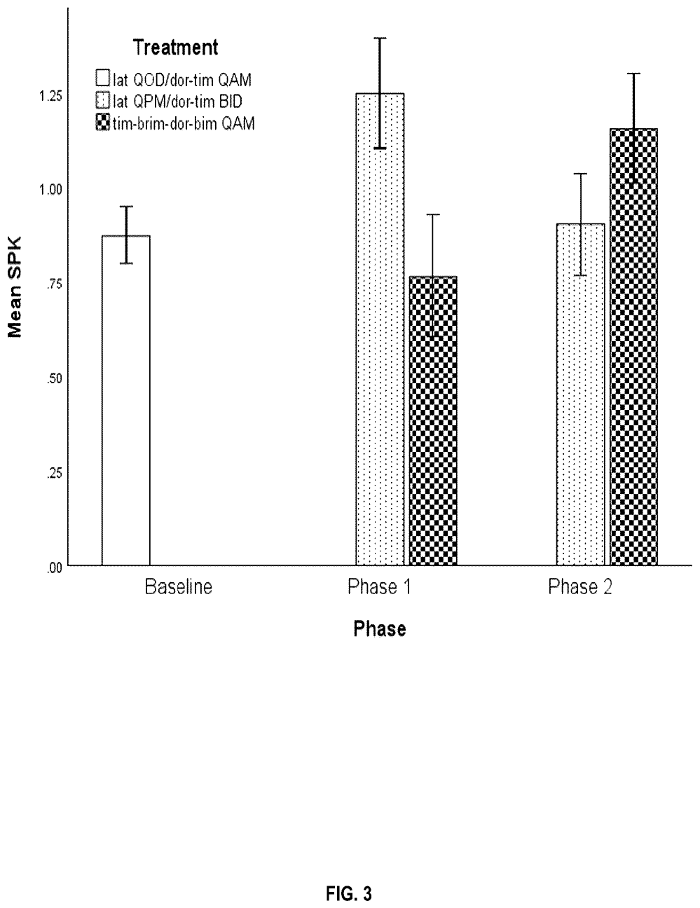

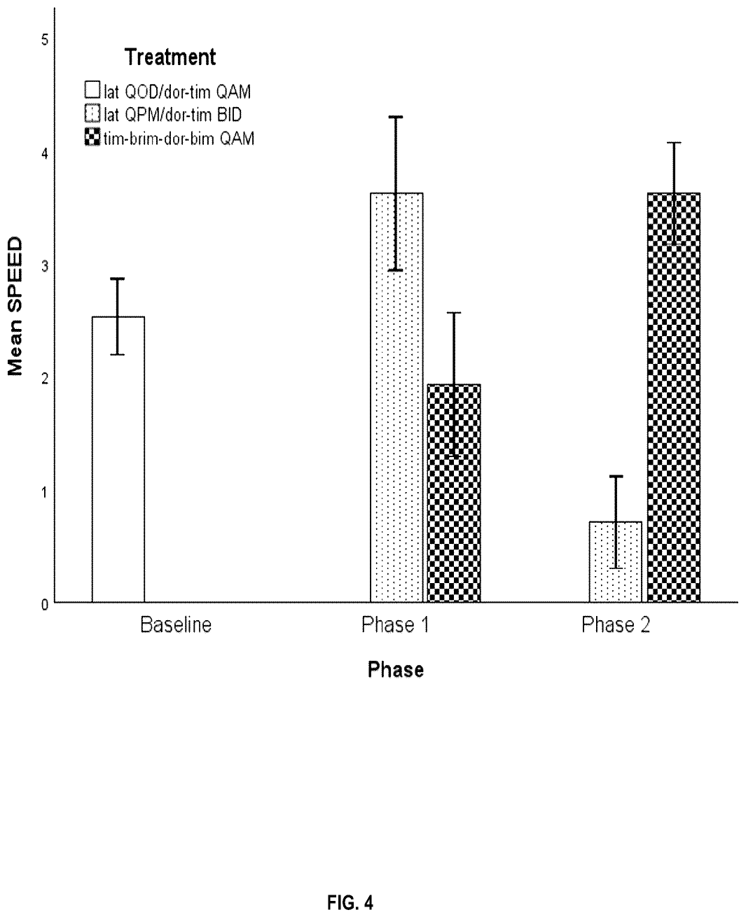

Secondary outcomes of the study included ocular surface disease, which was measured by evaluation of superficial punctate keratitis as depicted in FIG. 3, and the participants' survey using the SPEED questionnaire as depicted in FIG. 4. Upon averaging all visits, it was found that there were no significant differences between the three interventions. However, a sequence difference was discovered: those randomized to use the preserved medication first had higher overall ocular surface disease scores than those given preservative-free medication first (SPK, p=0.14; SPEED, p=0.04).

### Trial: Adverse Reactions

Only one allergic reaction to tim-brim-dor-bim QAM was diagnosed by conjunctival injection with eczematous and pruritic eyelids, but no serious adverse events were reported. Exacerbation of ocular surface disease (tearing, burning and itching) was reported by 3 participants: 2 found adequate relief with hot compresses and 1 withdrew from the trial.

### Adherence Survey

Of 100 patients surveyed, 68 had systematized their dosing. Most of those (55) volunteered that they were using a system without being asked. That is, when responding about the difficulty of QOD dosing, they replied to the effect that “it is not difficult, because | use the following system . . . ” The most popular system was a paper or electronic calendar. Statistics for the use of systems, missed doses, and difficulty of dosing are provided in Table 5.

TABLE 5
Every other day dosing: systems, missed doses, difficulty

| | Percentage | volunteered | | |
|---|---|---|---|---|
| | of total | use of a | missed a | reported dosing |
| System | (n = 100) | system | dose | to be difficult |
| No system | 32 | 0 | 10 (31%) | 13 (41%) |
| Paper or electronic | 32 | 26 | 2 (9.1%) | 0 (0%) |
| calendar | | | | |
| Odd calendar dates | 13 | 11 | 0 (0%) | 2 (15%) |
| Specific week days | 10 | 7 | 4 (40%) | 3 (33%) |
| Medicine moved | 13 | 11 | 2 (15%) | 1 (7.7%) |
| daily between 2 | | | | |
| locations | | | | |

Of the 100 patients surveyed, 81 judged alternate day dosing to be “not difficult”. It was found that patients without a dosing system were much more likely to report difficulty with QOD dosing (chi-squared p<0.001).

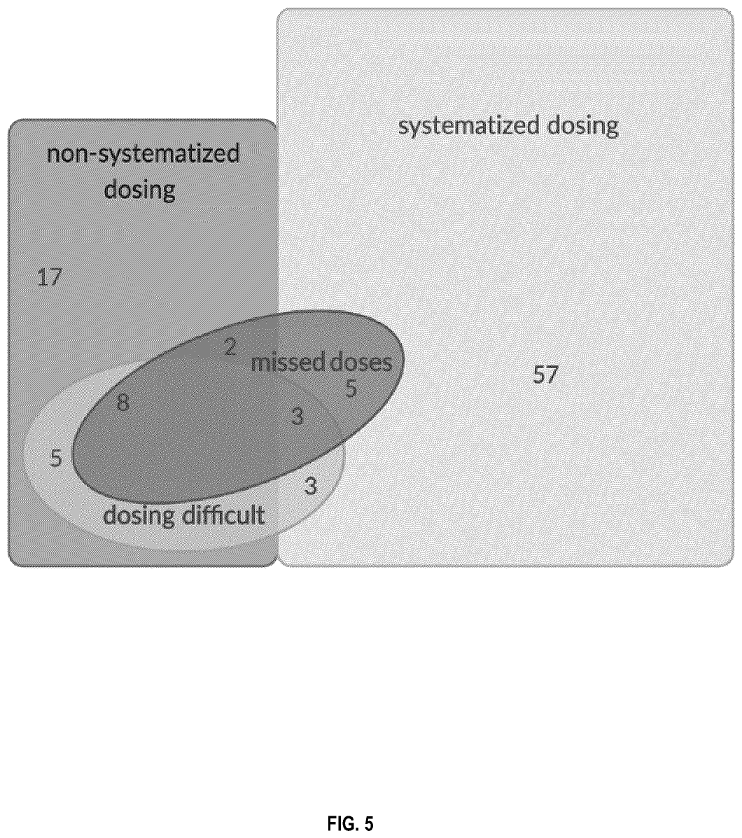

18 of the 100 patients reported not having used their medications on 2 consecutive days in the preceding month, which was also significantly associated with non-systematized dosing (chi-squared p=0.02). The Euler diagram depicted in FIG. 5 shows that most patients with systematized dosing reported neither regimen difficulty nor missed doses.

### DISCUSSION

It was primarily found that Lat QOD/dor-tim QAM and tim-brim-dor-bim QAM were both non-inferior to lat QPM/dor-tim BID. A strength of this study is that the double cross-over design coupled with duplicate IOP measurements by both Goldmann and pneumotonometry gave power for a clinically useful finding: it is 95% probable that halving the dosing frequency of lat QPM/dor-tim BID will raise diurnal IOP no more than 0.6 mm Hg and changing to a single daily drop of tim-brim-dor-bim would raise diurnal IOP no more than 0.4 mm Hg. This suggests that for patients taking latanoprost/dorzolamide-timolol, half-frequency dosing could reduce treatment burden without compromising efficacy. Because half of glaucoma patients have ocular surface disease, cutting the dose in half could help a vast number of patients. However, because these results apply to patients treated with SLT, it is not yet clear how these findings can inform the treatment of patients who lack access to sufficient medication or SLT.

It was surprising to find that the average IOPs of the trial subjects were so low. The treatment patterns likely explain the low IOPs: in practice, even patients with a mild glaucomatous visual field defect are typically treated with SLT and lat QOD/dor-tim QAM aiming for a target of <15 mm Hg. Patients with more advanced disease or higher IOPs are also typically prescribed brimonidine and would thus have been ineligible for the trial.

The finding that reduced dosing of 3 or 4 drugs yields IOPs indistinguishable from conventional dosing of 3 drugs accords with prior research. Studies of beta-blockers have shown no difference between once or twice daily dosing of timolol, and levobunolol. One study found that aqueous humor production did not begin to rise until 4 days after timolol was held. Among prostaglandins, bimatoprost 0.03% worked similarly whether dosed nightly or every other night-every other day dosing of travoprost raised IOP only 1 mm Hg at trough. For dorzolamide, a prior study of patients also taking latanoprost found average IOP the same whether dorzolamide was dosed 2 or 3 times daily, though the latter dosing gave 2.5 mm Hg lower IOP after the second dose.

Adherence to QOD dosing requires a little more thought than daily dosing, and conversations with physicians indicate that many would be reluctant to recommend such a regimen due to adherence concerns. However, the study survey (which was carefully phrased to normalize non-adherence) found that the great majority of patients systematized dosing and did not report difficulty with QOD dosing; missed doses and dosing difficulty were highly associated with failure to use a system. The most commonly used systems were a paper calendar or phone app reminder, dosing on odd dates (dosing consecutive days the 7 times a year when a month has 31 days), or dosing Monday-Wednesday-Friday-Sunday.

Furthermore, diminishing returns with additional ocular hypotensives is a documented phenomenon. Washout data from a prospective trial demonstrated that IOP rose 5.4 mm Hg with cessation of one medication, 6.9 mm Hg for two medications and 9.0 mm Hg for three medications. These data suggest that discontinuing one component of a triple regimen—as might be needed to improve adherence, comfort or cost—would typically raise IOP about 2 mm Hg. In contrast, the data from the present study provided that halving the dosing of a three-drug regimen did not raise IOP. As such, it appears that diminishing returns are more related to dosing than the number of agents. The concept that low doses of more agents are more effective than higher doses of fewer agents has already been validated in the cardiovascular literature: hypertensive patients were more likely to reach target blood pressure with low doses of quadruple or triple combination medications instead of higher doses of two medications. While further work is desirable, comparing the present results to preceding evidence suggests that reducing the frequency of dosing may be better for IOP than complete discontinuation of one agent.

It was also found that there may be a noxious effect from a high dose of preservatives that persists beyond a three-week respite using preservative-free medication, as indicated by the observed sequence effect upon SPEED scores. At baseline, subjects were using one to two preserved drops daily. Those who were subsequently randomized to use three preserved drops daily before the preservative-free treatment had worse ocular surface disease through the end of the trial.

The principal limitation of the present study was the lack of nocturnal IOP data. Unfortunately, collection of such data was logistically infeasible for this study. Beta-blockers and alpha-2 agonists are known to have no influence on IOP during sleep, but prostaglandins, SLT and carbonic anhydrase inhibitors do lower IOP during sleep. Therefore, a bedtime dose of carbonic anhydrase inhibitor would be expected to lower sleeping IOP and further study to clarify this issue would be useful. However, no study has ever shown twice daily dosing of timolol to be superior to once daily dosing, and it is also known that timolol has no effect upon IOP during sleep. Accordingly, the advantage of the second dose of dor-tim would be limited to the effect of dorzolamide alone.

Regulatory body trials of glaucoma medication typically exclude patients with prior SLT, pre-existing ocular surface disease, and test 1 or 2 medications; phase 1 trials which determine dosage last months, and phase 2 trials looking for side effects last a year. It is consequently difficult to conclude that dosing guidelines from such trials can be generalized to glaucoma patients with ocular surface disease who have been treated with SLT and are taking 3 or more medications for many years—the trial populations and treatment populations have very different characteristics.

A recent ophthalmic report found that wasted antibiotic drops at surgical centers cause significant carbon emissions, which is unsurprising given that healthcare accounts for 4.4% of greenhouse gas emissions worldwide and pharmaceuticals constitute 5% of those emissions. Because carbon loading of the atmosphere impacts health through wildfire, drought, flood, storm, crop failure, forced migration, and tropical disease, any opportunity to lower carbon emissions by using resources more efficiently merits consideration.

In conclusion, the data indicates that the dosing of a commonly used glaucoma regimen may be cut in half or replaced with a single daily compounded drop without changing diurnal IOP; these findings could help poor patients afford treatment and wealthier patients avoid ocular surface disease.

### Example 2

An exemplary pharmaceutical composition of the present disclosure was made by adding water for injection to a depyrogenated mixing vessel. Ingredients were then added, mixed, and dissolved sequentially as follow: sodium chloride, sodium citrate, polysorbate 80, poloxamer 407, timolol maleate, dorzolamide HCl, brimonidine tartrate, and then bimatoprost. The pH was measured and adjusted to 5.60 to 5.80 (target 5.70) using hydrochloric acid 37% or sodium hydroxide 25%. Water for injection was then added to the bulk to adjust the batch required mass specification. The contents were mixed for 40±5 minutes. The batch was then covered to be sterilized via filtration.

What is claimed is:

1. A method of treating glaucoma in a subject in need thereof comprising:
administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject in a single dose once per day.

2. The method of claim 1, wherein the subject is previously administered a single dose of the pharmaceutical composition twice per day or more, and further comprising reducing the frequency of the previous administration to once per day.

3. The method of claim 1, wherein the subject's mean intraocular pressure is statistically similar to the intraocular pressure of a patient treated with a pharmaceutical composition comprising latanoprost every other evening and a pharmaceutical composition comprising dorzolamide and timolol every morning.

4. The method of claim 1, wherein the subject's mean intraocular pressure is statistically similar to the intraocular pressure of a patient treated with a pharmaceutical composition comprising latanoprost every evening and a pharmaceutical composition comprising dorzolamide and timolol two times per day.

5. The method of claim 2, wherein reducing the frequency of administering the pharmaceutical composition to once per day raises the diurnal intraocular pressure by no more than 0.4 mm Hg.

6. The method of claim 1, wherein the pharmaceutical composition is preservative-free.

7. The method of claim 1, wherein the administration is topical administration via eye drops.

8. The method of claim 1, wherein the concentration of timolol or a pharmaceutically acceptable salt thereof in the pharmaceutical composition is about 0.5% w/v.

9. The method of claim 1, wherein the concentration of brimonidine or a pharmaceutically acceptable salt thereof in the pharmaceutical composition is about 0.15% w/v.

10. The method of claim 1, wherein the concentration of dorzolamide or a pharmaceutically acceptable salt thereof in the pharmaceutical composition is about 2% w/v.

11. The method of claim 1, wherein the concentration of bimatoprost or a pharmaceutically acceptable salt thereof in the pharmaceutical composition is about 0.01% w/v.

12. The method of claim 1, wherein the pharmaceutical composition further comprises sodium citrate, sodium chloride, polysorbate 80, poloxamer 407, and water for injection.

13. A method of treating glaucoma in a subject in need thereof comprising:
stopping administration of a pharmaceutical composition comprising latanoprost or a pharmaceutically acceptable salt thereof every evening and a pharmaceutical composition comprising dorzolamide or a pharmaceutically acceptable salt thereof and timolol or a pharmaceutically acceptable salt thereof two times per day; and
administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day.

14. The method of claim 13, wherein the subject is being administered the pharmaceutical composition comprising latanoprost or a pharmaceutically acceptable salt thereof every evening and the pharmaceutical composition comprising dorzolamide or a pharmaceutically acceptable salt thereof and timolol or a pharmaceutically acceptable salt thereof two times per day prior for the treatment of open-angle glaucoma prior to stopping the administration.

15. The method of claim 13, wherein the pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof is preservative-free.

16. The method of claim 13, wherein:
the concentration of timolol or a pharmaceutically acceptable salt thereof in the pharmaceutical composition is about 0.5% w/V;
the concentration of brimonidine or a pharmaceutically acceptable salt thereof in the pharmaceutical composition is about 0.15% w/v;
the concentration of dorzolamide or a pharmaceutically acceptable salt thereof in the pharmaceutical composition is about 2% w/v; and
the concentration of bimatoprost or a pharmaceutically acceptable salt thereof in the pharmaceutical composition is about 0.01% w/v.

17. A method of reducing patient burden during treatment for glaucoma in a subject in need thereof comprising:
stopping administration of a pharmaceutical composition comprising latanoprost or a pharmaceutically acceptable salt thereof every other evening and a pharmaceutical composition comprising dorzolamide or a pharmaceutically acceptable salt thereof and timolol or a pharmaceutically acceptable salt thereof every morning; and
administering a pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof to the subject once per day.

18. The method of claim 17, wherein the subject is being administered the pharmaceutical composition comprising latanoprost or a pharmaceutically acceptable salt thereof every evening and the pharmaceutical composition comprising dorzolamide or a pharmaceutically acceptable salt thereof and timolol or a pharmaceutically acceptable salt thereof two times per day prior for the treatment of open-angle glaucoma prior to stopping the administration.

19. The method of claim 17, wherein the pharmaceutical composition comprising timolol or a pharmaceutically acceptable salt thereof, brimonidine or a pharmaceutically acceptable salt thereof, dorzolamide or a pharmaceutically acceptable salt thereof, and bimatoprost or a pharmaceutically acceptable salt thereof is preservative-free.

20. The method of claim 17, wherein:
the concentration of timolol or a pharmaceutically acceptable salt thereof in the pharmaceutical composition is about 0.5% w/v;
the concentration of brimonidine or a pharmaceutically acceptable salt thereof in the pharmaceutical composition is about 0.15% w/v;
the concentration of dorzolamide or a pharmaceutically acceptable salt thereof in the pharmaceutical composition is about 2% w/v; and
the concentration of bimatoprost or a pharmaceutically acceptable salt thereof in the pharmaceutical composition is about 0.01% w/v.
